(19) United States
(12) Patent Application Publication
(10) Pub. No.: US 2007/0265551 A1
(43) Pub. Date: Nov. 15, 2007

(54) Method for delivering a catheter to a target in the brain of a patient and guide wire for a microcatheter for insertion in the brain of a patient

(75) Inventor: Marcus Pfister, Bubenreuth (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/789,229

(22) Filed: Apr. 24, 2007

(30) Foreign Application Priority Data
Apr. 28, 2006 (DE) 10 2006 020 402.6

(51) Int. Cl. A61M 25/00

(52) U.S. Cl. 600/585; 600/434; 604/264

(57) ABSTRACT

The movement of catheters and guide wires for catheters with the aid of magnets is known from the field of cardiology. Conventional magnets are, as a result of their size, not suited to being inserted into the brain of patient, because the vessels there are smaller than the cardiac vessels. To enable the navigation methods proven in cardiology also to be used in neurology, the invention provides a microcatheter having a guide wire, which features magnetic nanoparticles. The magnetic nanoparticles can be provided in the form of a ferrofluid or also as diamond nanoparticles in powder form. This enables navigation of the guide wire and thus ultimately of the catheter in the brain from the outside by way of magnetic fields.

### CROSS REFERENCE TO RELATED APPLICATIONS

This application claims priority of German application No. 10 2006 020 402.6 filed Apr. 28, 2006, which is incorporated by reference herein in its entirety.

### FIELD OF THE INVENTION

The invention relates to a method for delivering a catheter to a target, in particular in the brain of a patient. It also relates to a guide wire for a microcatheter for insertion in the brain of a patient as well as a microcatheter of this type.

### BACKGROUND OF THE INVENTION

The relative smallness of the cerebral vessels means that techniques which were developed for catheter applications in the heart are not used without restriction. As known for instance from the brochure “AXIOM Artis dFC Magnetic Navigation”, by Siemens Medical Solutions, 2004, Order No. A91 100-M1400-C914-1-7600, catheters in the cardiology field can be navigated by means of a changeable magnetic field. To this end, a small magnet is attached to the tip of the catheter. The catheter is inserted into the body of the patient. Magnetic fields are applied from outside the body of the patient, said magnetic fields exerting forces on the magnet. The magnet follows these forces and is thus moved (navigated) in the body of the patient. Catheters with guide wires are also known. The guide wire, which is easier to move than the catheter, is herewith initially guided to the destination, and the catheter is subsequently pushed after along the guide wire. During navigation with the aid of magnets, the guide wire must be equipped with a magnet (on its tip).

Conventional magnets require so much space that they can not be used in conjunction with catheters and guide wires, which are sufficiently small that they can be inserted in the brain of a patient (microcatheters with corresponding guide wires).

### SUMMARY OF THE INVENTION

It is the object of the invention also to provide a possibility for delivering the catheter to a target in a precise manner for applications in the brain.

The object is achieved by a method and a guide wire as well as a microcatheter as claimed in the claims.

In accordance with the invention, the method thus includes the provision of a catheter having a guide wire which can be moved in relation to the catheter body, with the guide wire comprising magnetic nanoparticles suspended in fluid and/or in powder form. Nanoparticles suspended in fluid are also known by the name “ferrofluid”. Other powder-type nanoparticles are diamond nanoparticles for instance, such as are described for instance in the article “Irradiation-Induced Magnetism in Carbon Nanostructures” by S. Talapatra, P. G. Ganesan, T. Kim, R. Vajtai, M. Huang, M. Shima, G. Ramanath, D. Srivastava, S. C. Deevi, and P. M. Ajayan in Physical Review Letters, Vol. 95, Article 097201 (2005) (see webpage http://www.wissenschaft.de/wissen/news/257612.html dated Apr. 27, 2006).

Such a catheter with the guide wire equipped according to the invention is then subsequently handled in such a way as is known from cardiac applications: the guide wire is introduced into the body of the patient, the guide wire is moved to the target by applying magnetic fields from outside the patient, which act on the magnetic nanoparticles (moved at least in sections), and the catheter body is then moved along the guide wire in order to reach the target in the brain of the patient.

The guide wire preferably features a diameter of less than 0.3 mm, preferably of less than 0.25, particularly preferably of less than 0.2 mm.

It preferably consists of soft (in other words pliable) plastic. Such a guide wire is known under the name of ‘floppy guide wire’. It is also referred to as flow-controlled, because the soft plastic enables the guide wire (at least on sections) to move in a manner controlled by the blood flow in the body vessels (in which the guide wire is located), i.e. to follow the blood flow (be carried along).

A guide wire for a microcatheter for insertion in the brain of a patient also forms part of the invention, to which or in which guide wire according to the invention magnetic nanoparticles are applied and/or arranged.

In the case of a plastic guide wire, the magnetic nanoparticles can be embedded into the plastic in any manner. The embedding can be granular. It is also possible for the magnetic nanoparticles to be held in a single receiving pocket, which either forms a core of the guide wire or is arranged on the exterior of the guide wire. In both cases, the receiving pocket can be bent together with the guide wire, because the nanoparticles do not provide any mechanical resistance to such pliability.

With the embodiment in which the magnetic nanoparticles are finely distributed over a front segment of the guide wire (in other words on the tip of the guide wire), provision can be made for said nanoparticles to be embedded into the plastic in a plurality of micropockets, i.e. to be held in receiving spaces in the plastic as in a sponge. The name ‘micropockets’ implies the provision of a large number of these pockets (100 to 10000 micropockets are conceivable, but also up to 1 million micropockets, with a plurality of nanoparticles being arranged in each micropocket).

The magnetic nanoparticles can be held (suspended) in a fluid (ferrofluid). The magnetic nanoparticles can also be diamond nanoparticles. These are usually present in the form of powder. It would also be possible to use nanoparticles to form a coating on the guide wire.

The invention also finds its expression in a microcathether having a catheter body and guide wire according to the invention.

### BRIEF DESCRIPTION OF THE DRAWINGS

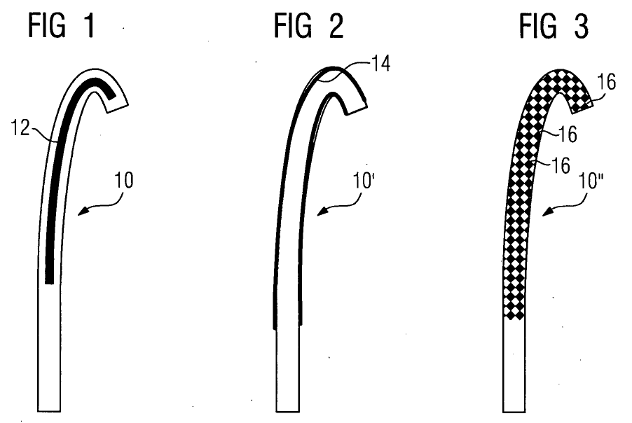

Preferred embodiments of the invention are subsequently described with reference to the drawings, in which FIG. 1 to 3 illustrate different embodiments of a guide wire according to the invention.

### DETAILED DESCRIPTION OF THE INVENTION

The use of guide wires for delivering a catheter body to a target is known. Guide wires consisting of pliable, soft plastic in particular are used in the brain. These guide wires have no metal element despite the name given to them historically. While conventional magnets would be too large to be fastened to guide wires for microcatheters, the invention provides the possibility of also equipping guide wires for microcatheters with a magnetic characteristic.

With a first embodiment illustrated in FIG. 1, provision is made for a guide wire made of plastic and denoted as a whole with 10, in the interior of which is located a single receiving pocket 12, which forms the core of the guide wire. Magnetic nanoparticles are arranged in the receiving pocket 12 and thus in the core of the guide wire 10. These can either be suspended in a fluid (as a ferrofluid) or they can be magnetic diamond nanoparticles, which have been introduced into the receiving pocket 12 in powder form. The pliability of the guide wire 10 is not significantly restricted either with the embodiment with the fluid or with the powder.

The guide wire 10 can be moved from the outside by means of magnetic fields, as is known for instance with guide wires for cardiac catheters in the prior art. For the basic design of a navigation system using external magnetic fields for the guide wire 10, the prior art can be adopted in its entirety. This also applies to the design of the external magnets specifically and to their activation. If need be, a size adjustment is required: The guide wire 10 features a diameter of 0.25 mm or even only of 0.1 73 mm for instance.

A conversion of the guide wire 10 from FIG. 1 is shown in FIG. 2 (guide wire 10′): Here the magnetic nanoparticles are arranged on the surface of the guide wire 10′ and form a receiving pocket 14 there. In future versions, it will certainly also be possible to do without a receiving pocket 14, if the magnetic diamond particles can be applied as a layer on the guide wire 10′. Techniques for generating such magnetic layers are currently being developed.

With a third embodiment, a guide wire 10″ is provided (FIG. 3), which comprises a large plurality of receiving pockets 16, in which the magnetic nanoparticles are arranged. The guide wire 10″ is then particularly formed to some degree like a sponge (in other words porously) in the region of its tip, being sealed from the outside.

1.-15. (canceled)

16. A method for moving a catheter comprising a guide wire to a target area of a patient, comprising:
arranging magnetic nanoparticles to the guide wire;
inserting the guide wire into a body of the patient;
moving the guide wire to the target area by applying a magnetic field from outside the patient acting on the magnetic nanoparticles; and
moving the catheter to the target area along the guide wire for performing a medical procedure of the patient.

17. The method as claimed in claim 16, wherein a diameter of the guide wire is less than 0.3 mm or 0.25 mm or 0.2 mm.

18. The method as claimed in claim 16, wherein the guide wire comprises a soft plastic so that the moving of the guide wire is controlled by a blood flow in body vessels of the patient.

19. The method as claimed in claim 16, wherein the magnetic nanoparticles are arranged in a fluid or a powder.

20. The method as claimed in claim 16, wherein the target area of the patient is in a brain of the patient.

21. A guide wire for inserting a microcatheter in a target area of a patient, comprising:
magnetic nanoparticles that are arranged to the guide wire.

22. The guide wire as claimed in claim 21, wherein a diameter of the guide wire is less than 0.3 mm or 0.25 mm or 0.2 mm.

23. The guide wire as claimed in claim 21, wherein the guide wire comprises pliable plastic and the magnetic nanoparticles are arranged in the plastic.

24. The guide wire as claimed in claim 21, wherein the magnetic nanoparticles are arranged in a single receiving pocket or a plurality of micropockets.

25. The guide wire as claimed in claim 24, wherein the single receiving pocket or the plurality of micropockets is a core of the guide wire or arranged on an exterior of the guide wire.

26. The guide wire as claimed in claim 21, wherein the magnetic nanoparticles are applied as a layer on the guide wire.

27. The guide wire as claimed in claim 21, wherein the magnetic nanoparticles are distributed over a segment on a tip of the guide wire.

28. The guide wire as claimed in claim 21,
wherein the magnetic nanoparticles are arranged in a fluid or a powder, and
wherein the fluid is a ferrofluid with the nanoparticles and the powder is diamond nanoparticles.

29. A microcatheter for inserting in a target area of a patient, comprising:
a catheter body; and
a guide wire that moves in relation to the catheter body and guides the catheter body to the target area, the guide wire comprising magnetic nanoparticles and moving to the target area by applying a magnetic field from outside the patient acting on the magnetic nanoparticles.

30. The microcatheter as claimed in claim 29, wherein the guide wire comprises pliable plastic and the magnetic nanoparticles are arranged in the plastic.

31. The microcatheter as claimed in claim 29, wherein the magnetic nanoparticles are arranged in a single receiving pocket or a plurality of micropockets.

32. The microcatheter as claimed in claim 31, wherein the single receiving pocket or the plurality of micropockets is a core of the guide wire or arranged on an exterior of the guide wire.

33. The microcatheter as claimed in claim 29, wherein the magnetic nanoparticles are applied as a layer on the guide wire.

34. The microcatheter as claimed in claim 29, wherein the magnetic nanoparticles are distributed over a segment on a tip of the guide wire.

35. The microcatheter as claimed in claim 29,
wherein the magnetic nanoparticles are arranged in a fluid or a powder, and
wherein the fluid is a ferrofluid with the nanoparticles and the powder is diamond nanoparticles.
